(19) United States
(12) Patent Application Publication
(10) Pub. No.: US 2018/0066271 A1
(43) Pub. Date: Mar. 8, 2018

(54) STABLE EPIGENETIC PLANT VARIANTS

(71) Applicants: University of Warwick, Conventry (GB); Max-Planck-Gesellschaft Zur Fórderung Der Wissenschaften E.V., Munchen (DE)

(72) Inventors: Claude Becker, Tuebingen (DE); Detlef Weigel, Tuebingen (DE); Jose Gutierrez-Marcos, Coventry (GB); Anjar Wibowo, Coventry (GB)

(21) Appl. No.: 15/703,085

(22) Filed: Sep. 13, 2017

Related U.S. Application Data
(63) Continuation of application No. PCT/EP2016/055377, filed on Mar. 11, 2016.

(30) Foreign Application Priority Data
Mar. 13, 2015 (GB) 1504309.4

(51) Int. Cl. C12N 15/82

(52) U.S. Cl. CPC C12N 15/8222; C12N 15/8205; C12N 15/8238

(57) ABSTRACT

The present invention relates to plants and the fields of agriculture and horticulture, with particular relevance to the area of plant improvement and plant breeding. More particularly, the invention concerns epigenetic variation in plants, and methods of capturing and stabilizing such variation in plants to be able to provide ranges of novel plant varieties and lines useful in plant improvement or in breeding programmes.

### CROSS-REFERENCE TO RELATED APPLICATIONS

The present application is a continuation of International Application No. PCT/EP2016/055377, filed on Mar. 11, 2016, which is entitled to priority to GB 1504309.4, filed Mar. 13, 2015, each of which is hereby incorporated by reference in its entirety.

### BACKGROUND

Classical plant breeding is a long established and well known field. Individual parent plants, selected for the various characteristics, including growth habit, productivity, resistance to disease or pests, or tolerance of e.g. drought, are crossed and the phenotypes and characteristics of the resulting progeny are assessed. Plants are crossbred to combine the traits from one line or variety with another. For example, a more rust resistant barley might be crossed with a less rust resistant but higher yielding variety. The desired outcome is a more rust resistant, yet still higher yielding barley variety. Progeny from the cross may then be backcrossed with the higher yielding parent to ensure the high yielding characteristic is not diluted. Inbreeding of progeny may be used to create varieties for further breeding purposes. Classical breeding therefore relies on the naturally occurring process of homologous recombination and the cross breeding ability of plants.

Techniques have been developed in order to expand ways in which genetic variability arises in plants. These include chemical mutagenesis using compounds such as ethane methanesulfonate (EMS) and dimethyl sulphate (DMS), or by using radiation. Other means of introducing genetic variation include harnessing transposons, which were first discovered in maize.

In parallel, techniques have been developed which allow the crossing of plants that would not naturally interbreed. Plant tissue culture can be used to rescue embryos from crosses that would otherwise not develop. Protoplasts may be fused together in an electric field, and viable recombinant cells regenerated in tissue culture. In another technique, embryonic cells from an interspecific cross that would be otherwise sterile are treated with colchicine, and plants are regenerated from this treated plant material.

More recently, molecular biology has added to the range of plant breeding techniques. Molecular markers or DNA fingerprinting are able to map thousands of genes simultaneously. Large populations of plants can therefore be screened more efficiently and without necessarily having to grow plants to maturity or to rely on visual identification of traits.

Another process involves making homozygous plants from a heterozygous parent that has all of the desirable traits. A problem with the diploid condition is that the process of homologous recombination during meiosis disrupts what may be desirable linkage between alleles/traits on the same chromosome. Conventional breeding takes about six generations of inbreeding to create homozygosity. By creating and crossing double haploids of chosen parental plants, homozygosity is achieved in one step, and the process of homologous recombination, which would otherwise be disruptive of trait linkages, is avoided; the outcome, however, is unpredictable and genotype-dependent.

There is also use of genetic modification, though there are restrictions in some parts of the world applying to commercial research and/or exploitation of this technology. A specific gene DNA sequence from one organism or plant can be introduced and expressed in the desired plant species or variety. For example, insect resistance is achieved by introducing a gene from Bacillus thuringiensis (Bt) encoding a protein that is toxic to some insects. Another example is herbicide resistance, e.g resistance to glyphosate, which can be achieved by introducing a glyphosate-resistant variant gene for 5-enolpyruvoyl-shikimate-3-phosphate synthetase (EPSPS).

In plants, most phenotypic variations, especially those of agronomic value such as drought resistance or growth rate, are continuously distributed and are often considered as quantitative traits. The genetic control of quantitative traits is often complex because of the large number of genes that are involved. Such traits can be very sensitive to environmental conditions. Because crop yield is known to be a quantitative trait, high accuracy and speed of identifying loci, genes and markers associated with quantitative traits are mandatory. Relevant loci of quantitative traits are localised by two basic approaches, linkage mapping and association mapping, based on the use of genetic maps and statistical analysis. Regions of the genome identified by linkage mapping are relatively narrow, but can still contain several hundred genes. Identification of genes underlying the quantitative trait loci requires positional cloning or direct tests of promising candidates. In contrast, association mapping checks directly the relationship between each polymorphism and the phenotypic trait variation in wild populations; here physical linkage and population structure are potential sources of false positives. Finally, one needs to confirm that an individual gene is responsible for the quantitative trait by using genetic or functional complementation.

Whole genome sequencing, targeting either the entire genome or genome-wide markers distributed at high density, is now being used in an acceleration of plant breeding programmes. For example, genotyping by sequencing (GBS) allows high throughput genotyping of a large number of SNP markers in a large number of individual plants. This is exemplified by genotyping of maize plants as described by Glaubitz, J. C., et al (2014) “TASSEL_GBS: A High Capacity Genotyping by Sequencing Analysis Pipeline” PLoS One 9(2): e90346.

Importantly, plants exhibit not only genetic sequence variability, but also a non-sequence variability known as epigenetic variation. Historically, the phenomenon was observed and studied in cell lineages within organisms. Meiotically or mitotically heritable changes in gene expression were found to occur independently of any changes in DNA sequence. Proposed mechanisms of epigenetic inheritance were mainly derived from mitotic cell studies. An emerging area of study relates to potentially meiotically inherited epigenetic changes in plants, whereby environmental triggers may result in a phenotypic response and an associated epigenetic change that may get fixed and passed on to subsequent generations. Hirsch, S. et al. (2012) “Epigenetic Variation, Inheritance, and Selection in Plant Populations” Cold Spring Harbor Symposia on Quantitative Biology, Volume LXXVII: 97-104 is a review article which collates and summarises research work in this area, yet concludes that there is still controversy about the existence of environmentally induced transgenerational epigenetic inheritance.

Gutzat R., et al. (2012) “Epigenetic responses to stress: triple defense?” Current Opinion in Plant Biology 15: 568-573 is another review article that identifies the connection between stress factors for plants and epigenetic responses to those stresses, but is guarded about whether epigenetic responses to stress are adaptive traits. Also, Boyko, A. & Kovalchuk, I. (2011) “Genome instability and epigenetic modification—heritable responses to environmental stress” Current Opinion in Plant Biology 14: 260-266 discusses how changes in genome stability and epigenetically mediated changes in gene expression may contribute to plant adaptation to the environment. This review lists, across a range of plant species, examples of environmentally induced transgenerational epigenetic effects that include the appearance of new phenotypes in successive generations of stressed plants. Similarly to the articles mentioned above, the authors' conclusion is that more studies are needed.

Roux, F., et al (2011) “Genome-Wide Epigenetic Perturbation Jump-Starts Patters of Heritable Variation Found in Nature” Genetics Vol 188, 1015-1017. This scientific paper reports on a study of Arabidopsis plants looking at DNA methylomes and comparing populations experimentally perturbed and those of natural populations. Alterations in DNA methylation were found to be heritable and there was surprising similarity in heritability patterns between experimental and natural populations of plants.

Stroud, H., et al (2013) “Plants regenerated from tissue culture contain stable epigenome changes in rice” eLIFE (2013) 2:e00354. DOI: 10:.7554/eLife.00354. This scientific paper reports on how DNA methylation in rice is lost when subjecting plant material to tissue culture. Rice plants regenerated from tissue culture were found to have lost epigenetic character.

Cortijo, S., et al (2014) “Mapping the epigenetic basis of complex traits” Science Vol 343 1145-8 describes the analysis of a population of isogenic Arabidopsis lines that segregate experimentally induced DNA methylation changes at hundreds of regions across the genome. Several differentially methylated regions (DMRs) were found to be epigenetic quantitative trait loci (QTL(epi)), and were reproducible and susceptible to artificial selection.

Kooke, R., et al (2015) “Epigenetic Basis of Morphological Variation and Phenotypic Plasticity in Arabidopsis thaliana” Published online before print February 2015, doi: http://dx.doi.org/10.1105/tpc.114.133025. This study on Arabidopsis finds extensive heritable epigenetic variation in plant growth and morphology in neutral and saline conditions. The variation in plasticity is associated significantly with certain genomic regions in which the ddm1-2 inherited epigenotypes caused an increased sensitivity to environmental changes. Many QTLs for morphology and plasticity overlap. The authors suggest epigenetics contributes substantially to variation in plant growth, morphology, and plasticity, especially under stress conditions.

Heterosis is a most important aspect of plant breeding. It circumscribes the often observed phenotypic superiority of a hybrid plant compared to its genetically distinct parents with respect to traits such as biomass, growth rate and yield. The phenomenon has been exploited successfully for many years for many plant species, but the molecular basis of heterosis remains elusive. He, G et al (2013) “Epigenetic Variations in Plant Hybrids and Their Potential Roles in Heterosis” Journal of Genetics and Genomics 40: 205-210 is a review article which postulates relationships between DNA methylation, microRNAs (miRNAs), small interfering RNAs (siRNAs) and histone modifications on the one side and heterosis on the other. Comparative analyses of parents and hybrids suggest that there may be some associations between changed chromatin states and gene activity in hybrids, and that this may equate with heterosis. However, the authors clearly state that the available evidence is well short of that needed to show epigenetic variation as the source of heterosis, and that no particular molecular mechanism is proven.

Heterosis was studied at the single-base-pair resolution level of DNA methylomes in Arabidopsis thaliana, reported in Shen, H. et al., (2012) “Genome-Wide Analysis of DNA Methylation and Gene Expression Changes in Two Arabidopsis Ecotypes and Their Reciprocal Hybrids” The Plant Cell 24: 875-892. DNA methylation of parental lines was compared to that in the respective hybrids. Both hybrids displayed increased methylation compared to the parents, especially in transposon regions, and 77 different genes were associated with methylation changes. The growth vigour of the F1 hybrids was compromised by treatment with a DNA-demethylating agent, suggesting that genome-wide remodelling of DNA methylation may play a role in heterosis.

At least three research groups have described and studied certain plant embryonic transcription factors, their roles in plant embryo growth and development, and how these can be used to induce somatic embryogenesis. Köszegi, D. et al. (2011) “Members of the RKD transcription factor family induce an egg cell-like gene expression program” The Plant Journal 67: 280-291 isolated two RWP-RK domain-containing (RKD) factors from wheat, where they are preferentially expressed in egg cells. The Arabidopsis genome has five of these RKD genes and these were studied in a more detailed functional analysis. Two of these, AtRKD1 and AtRKD2, were preferentially expressed in the egg cell. The AtRKD4 gene was expressed mainly in tissues containing reproductive organs, but ectopic expression of this gene produced no discernible phenotype. Transient expression of an AtRKD4-GFP fusion in protoplasts showed localisation of the protein in the nucleus.

In Jeong, S., et al. (2011) “The RWP-RK Factor GROUNDED Promotes Embryonic Polarity by Facilitating YODA MAP Kinase Signalling” Current Biology 21: 1-9, the function of the GROUNDED gene (GRD), also known as RKD4 (see above), is explained as being that of a transcription factor which promotes the elongation of the zygote and the development of its basal daughter cell into the suspensor.

Waki T et al (2011) Current Biology 21, 1277-1281 reports on observational experiments that describe and then explain how the gene RKD4 controls and affects gene expression in early plant development. The gene is one of the RWP-RK genes in Arabidopsis. Two mutant alleles of the RKD4 gene, rkd4-1 and rkd4-2 were initially found to be associated with germination defects, whereby the seed germinated but the roots did not form properly. The spatiotemporal expression of RKD4 was investigated using a two-component reporter construct in which the RKD4 promoter drives a GAL4:VP16 (GV) transcriptional activator of GFP. Expression of the RKD4:GFP fusion in this two-component system completely rescued the rkd4-1 mutant. Based on sequence similarity, the RWP-RK proteins and therefore RKD4 is suggested to encode a transcription factor and is hypothesised to be a regulator of early embryogenesis. In a dexamethasone-inducible, RKD4-overexpressing transgenic Arabidopsis line, the ectopic RKD4 expression resulted in upregulation of a number of genes, some of which were identified as being early embryo specific. Longer induction of RKD4 overexpression was found to trigger somatic embryogenesis. RKD4 is therefore proposed to directly or indirectly promote expression of genes needed for initiating the patterning process in the zygote and early embryo.

WO2007/073221 (Instytut Hodowli I Aklimatyzachji Roslin (Plant Breeding and Acclimatization Institute)) relates to in vitro culture of plant materials, where phenotypic and/or genetic variation arises as a consequence of the culturing process; the phenomenon of so-called somaclonal variation. Some of this is thought to be due to genetic changes and some due to epigenetic changes. Disclosed is a method of quantitative and qualitative genetic fingerprinting of induced variability in plants, e.g. double haploid barley regenerated from in vitro microspores. The disclosed method estimates the respective percentages of sequence-related and methylation-related variability stably passed on to plant progeny. The particular genetic fingerprinting method is that of Amplified Fragment Length Polymorphism (AFLP). Selected primer pairs are used to discriminate presence and absence of methylation at the cleavage sites. By following and comparing the fingerprints of plant material having undergone different paths in the in vitro culturing and regeneration process, some of the nature and extent of stable somaclonal variability can be described.

A key question in plant breeding remains: how can one manipulate quantitative traits that are stably transmitted to successive generations without introducing undesirable chromosomal changes? Whilst genetic lesions or chemical treatments, including hormone treatments, can induce heritable changes associated with novel traits in the epigenomic landscape of plants, these changes are also accompanied by undesirable side effects, such as poor heritability or genetic changes that affect plant viability. Ways are needed to identify, control and remove such undesirable side effects and increase the efficiency and speed with which plant breeding programmes can be pursued.

### BRIEF SUMMARY OF THE DISCLOSURE

The inventors have discovered that by transforming somatic plant tissues or cells to express zygotic or embryonic transcription factors, and then regenerating plants from those transformed tissues or cells, the resulting plants retain substantially or entirely the epigenetic characteristics of the originally transformed parent plant cells. Further, the inventors have found that the epigenetic characteristics of these plants are substantially or entirely stably and heritably maintained.

In essence, the inventors have learned that by reprogramming somatic plant cells from different tissues or exposed to different conditions into a zygotic or early embryonic state, these cells can then be regenerated into fertile plants that retain the epigenetic characteristics of the somatic cells prior to the reprogramming.

The inventors therefore provide methods, materials and a system for generating stable and transgenerational epigenetic plant varieties, thereby avoiding the undesirable side effects accompanying the known methods involving chemical or hormone treatments.

Additionally, the inventors have discovered that subjecting parental plants to stress conditions prior to reprogramming, be it abiotic stress such as cold, heat, salt or aluminium, or biotic stress such as bacterial pathogens or fungi, expands the resulting range of stable and heritable epigenetic variants and their associated phenotypic traits. The inventors therefore provide methods, materials and a system for quicker and more efficient generation of an extensive range of plant variants for breeding purposes.

Accordingly, the present invention provides a method for generating stable, heritable epigenetic configurations in plants, comprising:

a. introducing into a parent plant or plant material an expression construct that comprises a nucleic acid sequence encoding a zygotic and/or an embryonic transcription factor;
b. controlling the time of expression of the transcription factor in the plant or plant material for a period of time; then
c. growing transformed plant material in culture without expressing the transcription factor;
d. identifying a regenerant plant initial, plant embryo or plantlet; and
e. growing the plant initial, plant embryo or plantlet into a mature progeny plant.

The introduction of expression constructs is preferably by way of transformation. Additionally or alternatively, expression constructs may be inducible expression constructs to permit controlling of the time of expression of the transcription factor.

The stability of an epigenetic plant variety produced as a result of the method of the invention is such that the measurable traits of the progeny plant are transmissible via reproductive processes that can be natural or a consequence of human technical intervention. For example, when the reproductive process is a natural one it may be by selfing or by crossing with another plant, so that they are passed on into at least the next generation of plants without substantial diminution or loss, qualitatively and/or quantitatively. The epigenetic traits that are passed on may be quantitative, observable and/or measurable, and/or they may be based on molecular markers, and/or on gene expression profiling. The stability may be such that they persist from not just the first generation, but preferably to the second, third, fourth or fifth or more generations.

The epigenetic basis of a phenotype, i.e. traits, of the plant may be ascertained from methylation analysis, optionally on a whole-genome profiling basis. Additionally or alternatively, the epigenetic character of a plant may be defined on another basis such as histone modification (chromatin remodelling), e.g. via the likes of histone methylation, acetylation, phosphorylation, ubiquitination, glycosylation, ADP-ribosylation, sumoylation, deamination and proline isomerization. Additionally or alternatively a phenotype may be characterised based on observation, counting and or measurement of plant traits as will be well known to a person of average skill in the art, e.g. biomass, yield, height, drought tolerance, saline tolerance, flower colour, etc.

The expression of the transcription factor by the inducible expression construct occurs during a period of time sufficient to achieve a reprogramming of the cells of the parental plant to a zygotic or embryonic stage, but which does not significantly disrupt or alter the epigenetic characteristics nor the traits or phenotype of the plant governed by the epigenetics of the genome. Whilst not wishing to be bound by any particular theory, the inventors believe that a genetic reprogramming of the plant cell back to a zygotic or early embryonic state resets the genetic developmental clock, but does not adjust or substantially alter the sequence or the epigenetic character of the plant tissue or cell genome. The period of time of expression of the transcription factor may be empirically determined, but can be a period measured in hours and/or days.

If a period of hours, this may be from 1 to 24 hours, e.g. 2 to 24 hours; 3 to 24 hours; 4 to 24 hours; 5 to 24 hours; 6 to 24 hours; 7 to 24 hours; 8 to 24 hours; 9 to 24 hours; 10 to 24 hours; 11 to 24 hours; 12 to 24 hours; 13 to 24 hours; 14 to 24 hours; 15 to 24 hours; 16 to 24 hours; 17 to 24 hours; 18 to 24 hours; 19 to 24 hours; 20 to 24 hours; 21 to 24 hours; 22 to 24 hours; or 23 to 24 hours. If a period of days, then the number of days may be in the range 1 to 12 days; optionally 1 to 11 days; 1 to 10 days, 1 to 9 days, 1 to 8 days; 1 to 7 days; 1 to 6 days; 1 to 5 days; 1 to 4 days; 1 to 3 days; or 1 to 2 days.

The period may be a combination of days and hours, wherein the number of days is as defined above and is combined with a number of hours as defined above.

A person of average skill will understand that for functionality, the expression construct comprises necessary genetic elements needed to achieve expression of the transcription factor in the transformed cell for the period of time. A suitable promoter is required which is under a control, directly or indirectly, of an exogenous factor.

In some methods of the invention, parent plant tissue including individual cells are transformed in the whole plant context and then at some stage after transformation, transformed tissues and/or cells are isolated from the parent plant. The step of isolating the tissue and/or cells may take place before, during or after expression of the transcription factor for the period of time.

The introduction of the expression construct into the parental plant or plant material is preferably by way of a transformation of plant cells. Transformation vectors of use in the invention are well known to a person of skill in the art. Particularly well known and preferred are Agrobacterium binary Ti vectors, for example. Other methods of transformation may be used to introduce the expression construct into the parental plant or plant material, either alternatively or in addition to the above and to each other, including ballistics, polyethylene glycol treatment or microinjection.

The transformation of the tissue and/or plant cells with the expression construct may be a transient transformation, for a period of time sufficient to allow inducible expression of the zygotic and/or embryonic transcription factor.

In other methods of the invention, organ or tissue and/or cells are isolated from the parent plant prior to transformation with the expression construct.

The expression construct may be an inducible expression system, preferably wherein the induction is selected from alcohol, tetracycline, steroid, metal or a pathogenesis related protein. For example, the system may be AlcR/AlcA (ethanol inducible); GR fusions, GVG, and pOp/LhGR (dexamethasone inducible); XVE/OlexA (β-estradiol inducible); or heat shock induction. The expression of the transcription factor is thereby inducible for the period of time. In a particularly preferred embodiment, a two-component dexamethasone inducible system is used.

Transformed plant material, such as organs, tissue or cells, are preferably contacted with the chemical inducer for the period of time, following which the tissue or cells are transferred to a regeneration medium lacking the chemical inducer.

In the preferred methods of the invention, prior to step (b) a whole parent plant from step (a) is grown to reproductive maturity, selfed and then grown on to set seed, wherein the plant material of step (b) is the or a seed. In this context a seed is therefore a plant material as referred to herein.

In the preferred embodiments, the transcription factor is selected from: RKD4, BBM, LEC2 and FUS3. This also represents a preferred order of effectiveness of transcription factors, with RKD4 being the most preferred. A combination of one or more of these transcription factors or any other zygotic or embryonic transcription factor may be used in accordance with the invention.

Before and/or after transformation, isolated tissue or cells may be sorted, optionally according to one or more of tissue or cell type, protein marker, methylation profile or gene expression profile. Cells can be sorted according to tissue type and then further by methylation profile and individual cells used in the cloning. Resulting plants will generally have the same methylation profile as the mother cell, although some variation will naturally occur within the plant.

Tissues and/or cells may be identified by using dyes or specific markers, particularly fluorescent markers such as GFP, ECFP, EGFP, EYFP, Venus YFP, DsRed., RFP1 or mCherry. A GAL4-UAS system may be used to achieve desired cell and/or tissue specific expression of the fluorescent marker. Various methods of fluorescence microscopy may be used, e.g. confocal laser scanning fluorescence microscopy, spinning disc confocal microscopy, multiphoton microscopy or widefield fluorescence microscopy. Tissues and or cells can be isolated by microdissection. In other methods, protoplasts may be prepared from the transformed plant cells and labelled with fluorescent labels to allow for fluorescence activated cell sorting (FACS) of the protoplasts.

In the method of the invention, tissue or cells may be obtained from the root of the parental plant and these have found to be a preferred and wide ranging source of stable and heritable epigenetic variation. Other tissues such as shoot, leaf, flower or fruit may be used as the starting material for transformation and expression of zygotic and/or embryonic transcription factors in accordance with the invention. Also included within the scope of the method of the invention are plant organs or explants, as well as tissues and/or cells. Reference to tissues and/or cells herein thereby includes any plant part which can be transformed with the expression vector comprising the transcription factor and which can then be transiently caused to express the transcription factor, followed by growing the plant part or organ, tissue or cell in culture so that plant regeneration by a process of embryogenesis can take place.

Advantageously, a parent plant or plant part may be subjected to a stress condition prior to transformation and expression of the zygotic or embryonic transcription factor. This may produce up to about 100 times, potentially 400 times or more, as many epigenetic variants by the method of the invention than by relying on the natural (i.e., unstressed) methylation profile of the plant alone.

The stress condition may be abiotic and/or biotic, and may comprise one or more of such abiotic and/or biotic stress conditions. Again, without wishing to be bound by particular theory, the inventors believe that a range of epigenetic differences can arise as between individual cells and/or tissues in a plant due to growth, development and stress history. Therefore a whole plant may be expected to be a chimera in terms of its epigenetic character. The separation and isolation of such cells and tissues so that they can be used as the starting material for regenerating fresh plants allows plants of homogeneous epigenetic character throughout their tissues and cells to be provided.

Abiotic stress, for example, may include photoperiod, where the plant is moved from one photoperiod to another. This could be moving from long days to short days or vice versa. This could also be moving the plant from a light:dark cycle to an entirely light phase, or entirely dark phase before the step of transformation and expression of the transcription factor in accordance with the invention. Another example is temperature, where the plant is moved from a starting temperature to a higher or a lower temperature as a step change, or as a graduated or gradual continuous change. The temperature might be cycled between a maximum or a minimum. At an extreme the temperature could be below freezing to mimic frost. Other abiotic stresses may include physical stress such as wind pressure or physical pressure applied to the plant resulting in bending, and this may be as a dynamic or a static application of physical force. Another example is water stress where the plant may be denied entirely of water so that it is so stressed that it wilts. The water stress may be less severe such that the stomatal response of the plant prevents wilting but reduces transpiration. A further example of stress is salinity, whereby the salt concentration of the medium in which the plant is rooted is altered, usually by way of increase.

Biotic stress may also be applied, for example insect pest attack, or fungal or bacterial pathogen attack.

Plainly a number of different stress conditions may be applied to a plant sequentially or simultaneously. The stresses may be applied repeatedly at different stages of development, for greater or lesser periods of time.

The epigenetic character of the plant may be manifested as a change in at least one quantitative trait, such as plant height, crop yield, disease resistance, flower colour and shape, i.e., traits that have continuous, unbroken quasi-normal distributions in a population.

The epigenetic character of the plant may be identifiable by its DNA methylation and histone modification profile (i.e. histone marks). This may be carried out in a variety of ways, whether specifically targeted to loci or regions of the genome, or as part of whole genome sequencing. For example, Illumina provides equipment and consumables and software for sequencing-based DNA methylation analysis such as whole-genome busulfite sequencing (WGBS) or reduced representation bisulfite sequencing (RRBS).

The growing of transformed tissues and/or cells in culture employs well known culture media and methods. Often particular media compositions and particular protocols for growing plant tissues and/or cells in culture have already been established as providing optimal growth conditions. Sometimes these may be species-specific, but a person of average skill will be readily able to provide the necessary culture media and protocols from textbooks and the scientific literature. (See for example: “Plant Cell and Tissue Culture” A Tool in Biotechnology Basics and Application: Eds: Neumann, K.-H. & Imani, J. Spinger (2009).)

The culture of plant tissues and/or cells may be in solid or in liquid media, as is most appropriate and desirable. Organs and explants may be grown on solid media. When cells are cultured they may form a callus culture.

Plant parts, organs, tissues or cells in accordance with the invention are grown in culture for a period during which expression of the transcription factor takes place, following which the culturing continues in the absence of such expression. If the expression is induced chemically then plant material is grown in the absence of the inducer, which in practice may mean that the plant material may need to be transferred to a fresh culture medium.

Following cessation of transcription factor expression (usually corresponding to withdrawal of the inducer; i.e. a transfer to fresh medium), plant material is continued to be grown in culture for a sufficient time and under conditions conducive to plant regeneration, preferably via embryogenesis. So, in accordance with the invention, plant initials, embryos or plantlets that form from the cells and/or tissue being grown in culture are, ideally but not necessarily, visually identified. At some convenient stage, embryonic plant material, embryos or plantlets are then isolated from the originating tissue or cell culture and grown on to the stage of mature plant, being a plant which is sexually mature in that it has produced ovule(s) and/or pollen.

So, also in accordance with other aspects of the invention, a mature progeny plant from the above process may be used itself as a parent in a process of producing a second generation of progeny plants that have substantially the same epigenetic character as the first generation. An ovule of the mature progeny plant may be fertilized with pollen of the same plant and the plant grown on, so that it can set seed.

Seed of the above plant may be collected, germinated and then a resultant seedling is grown into a mature plant. This then is a third generation progeny plant.

The above process of selfing may be continued any number of times; optionally 2, 3, 4, 5 or more times to produce fourth, fifth, sixth, etc. generation progeny plants. The stable and heritable (transgenerational) nature of the epigenetic character of the first generation progeny is determinable by a process involving sexual reproduction, preferably inbreeding, but most by selfing. Other ways of making subsequent generations of progeny plants starting with the first are available, including backcrossing with the first generation progeny. A mixture of selfing and backcrossing may be used. In such ways, individual plant lines or varieties of stable and heritable epigenetic character are provided.

The invention therefore includes a stable, transgenerational (i.e. heritable) epigenetic plant variety obtained according to any method as hereinbefore described.

Also forming part of the invention is germplasm of such stable epigenetic plant varieties. Germplasm may conveniently be in the form of seed, but also included is pollen or ovules.

The invention also includes any plant biomass or plant material of stable epigenetic plant varieties of the invention, including leaves, fruit, seed, stems or woody parts. Therefore also included are any plant products involving some form of processing, for example, flour, meal, dried leaves, timber. Diagnostic testing of samples of such plant products where polynucleic acid remains present may permit identification of the particular plant species and epigenetic strain of that species.

Stable epigenetic plant varieties of the invention having desired phenotypic traits are therefore useful to incorporate into a plant improvement or breeding program. The invention therefore includes a method of plant breeding comprising combining stable transgenerational epigenetic material of a first parent plant with genetic material of a second parent plant. The second parent plant may also be an epigenetic variety of the invention, or it may be another variety or line that has no uniform or stable epigenetic character.

The combining of genetic material may make use of naturally occurring sexual reproduction processes in plants, or it may involve artificial means, e.g. protoplast fusion.

Once a stable epigenetic plant variety or line is available, this plant and its genetic material and germplasm may be incorporated into any classical or molecular breeding programme with the aim of producing further new varieties which may be themselves hybrids.

Accordingly the invention also provides in vitro methods of plant improvement comprising combining stable epigenetic genetic material of a first parental plant with genetic material of a second parental plant. In preferred aspect, the first parental plant, or the first and the second parental plant are produced according to a method as hereinbefore described.

Advantageously the invention helps in providing large numbers of stable epigenetic plant lines, each of a different epigenetic character, thereby assisting in speeding up the development of new plant varieties and hybrids by making an increased pool of stable and heritable phenotypic variants available more quickly for selection and crossing.

The invention also includes plants or plant cells, tissues, organs or parts, obtainable by any of the methods of the invention as described herein.

The invention further includes a plant-derived product obtained from any plant obtained or obtainable in accordance with any method of the invention, as described herein. For example, a processed plant product produced by a process of milling or grinding, e.g. flour or meal. Such plant products, whilst not possessing identifiable whole cells, may include sequenceable genetic material such that the particular plant strain or variety can be determined. The presence of particular unique nucleotide sequence tags, if incorporated into the genome of the originating plants, will thereby allow for identification of origin and tracking of a processed plant product, as may be desired.

### BRIEF DESCRIPTION OF THE DRAWINGS

Embodiments of the invention are further described hereinafter with reference to the accompanying drawings, in which:

FIG. 1, comprising FIG. 1A and FIG. 1B, depicts methods of regenerating Arabidopsis plants. FIG. 1A shows a first method of regenerating Arabidopsis plants from dissected and isolated tissues from transformed root and shoot. FIG. 1B shows a method of regenerating Arabidopsis plants from de-differentiated cell cultures. Embryonic cell culture is carried out after controlled RKD4 expression.

FIG. 2 is a heat map showing methylation differences found in leaf and root tissues of plants that have been regenerated from different cell types.

FIG. 3 is a diagram showing the result of a principal component analysis showing DNA methylation differences present in leaf and root tissues of plants regenerated from different cell types.

FIG. 4 is a heat map of unsupervised cluster analysis of whole-genome gene expression of plants regenerated from root (RO) and leaf (LO) tissues.

FIG. 5 is a bar chart of data from phenotypic analysis of parents and regenerants subjected to biotic stress.

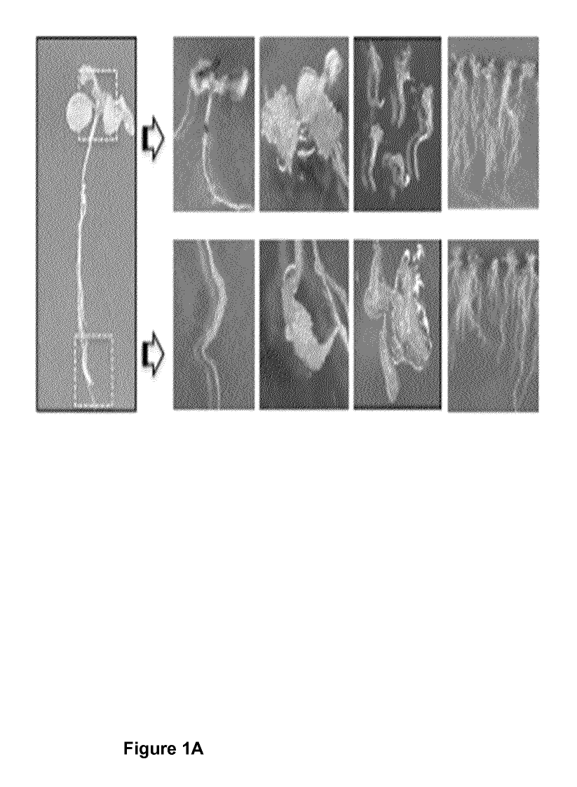

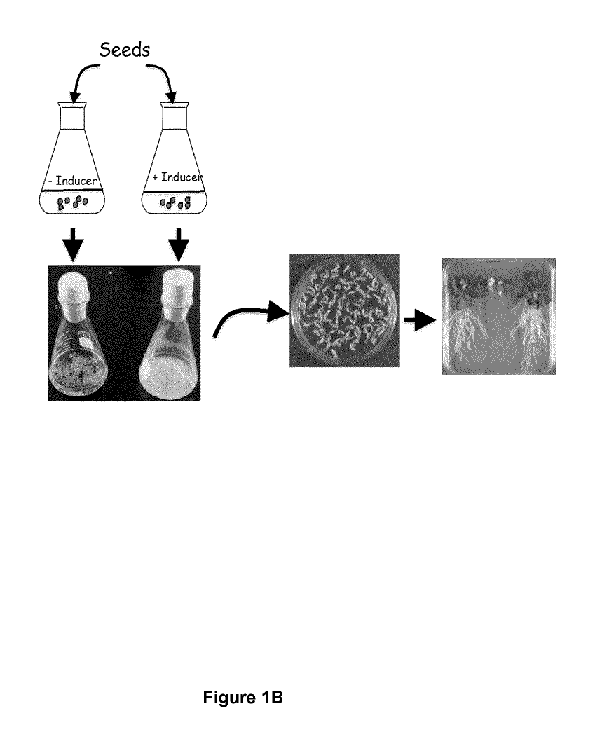

### DETAILED DESCRIPTION

The inventors have used short read sequencing of bisulfite treated genomic DNA and computational analysis to show how a range of epigenetic variation exists between cells in a plant. Also that when an individual plant cell of particular epigenetic character is reprogrammed back to a zygotic or an embryonic developmental stage and regenerated into a plant, that this epigenetic character is stably maintained through more than one generation. Also, qualitatively, the differences in gene expression due to differing methylation, particularly compared to the parent plant, concern genes associated with plant growth regulation or pathogen resistance. Such variation in gene expression has also been shown to manifest itself in advantageous growth of regenerant variant plants in response to particular biotic stress compared to parent controls.

So the inventors have discovered that epigenetic variation in the form of methylation patterns as between somatic cells of plant tissues can be fixed upon cell culture and regeneration of plants, and the individual variants remain fixed into subsequent generations of plants when derived from a single cell.

Using transgenic plant lines carrying zygotic transcription factors, the inventors tested a range of transcription factors, including RKD4, BBM, LEC2 and FUS3. An established two-component inducible expression system was used, activated upon exposure to the chemical inducer dexamethasone. Once somatic plant cells are reprogrammed into undifferentiated initials by the effect of expression of the transcription factor, new plants are recovered simply by growing the transformed and induced cells or tissue in solid culture media, in the absence of the inducer. New plant materials regenerated from this are then grown over three generations (selfing) and assessed in their genetic and epigenetic structure. The analysis shows that whilst no changes in gene sequence take place, the regenerated plants have extensive epigenetic variation. What is more, this epigenetic variation is found to be stable over several generations. A technical advantage of this is that by generating a large number of epigenetic lines or varieties, the inventors provide a useful source of highly characterised, genetically known and stable functional diversity in plants.

Collectively, the approach now allows the generation of an unlimited source of epigenetic variation in plants, avoiding the hitherto undesirable effects of using genetic lesions or chemical treatments in the known methods.

The methods and epigenetic plant variants of the invention have wide applicability for plant breeding efforts and biotechnology research. What is now possible is to generate rapidly many new novel phenotypic variants that are epigenetically stable over many generations, but do not change the genetic structure. Stable epigenetic plant lines can be provided to improve resistance to pathogens and environmental stress, increase yield and enable the production of new metabolic compounds. Another useful application of the invention is in the propagation of recalcitrant plant material (e.g. endangered species or species difficult to reproduce through standard seed propagation).

Some advantages over the traditional methods of breeding and genetic modification (GM) are the speed and the range of epigenetic plant varieties of known and uniform genotype that can be achieved.

The methods of the invention are applicable to any plant from any plant taxonomic group, including angiosperms or gymnosperms. Amongst the angiosperms the invention is applicable to monocots and dicots.

The invention is applicable to any angiosperm plant species, whether monocot or dicot.

Preferably, plants which may be subject to the methods and uses of the present invention are crop plants such as cereals and pulses, maize, wheat, potatoes, tapioca, rice, sorghum, millet, cassava, barley, pea, and other root, tuber or seed crops. Important seed crops are oil-seed rape, sugar beet, maize, sunflower, soybean, and sorghum. Other plants to which the present invention may be applied may include lettuce, endive, and vegetable brassicas including cabbage, broccoli, and cauliflower, and carnations, geraniums, tobacco, cucurbits, carrot, strawberry, sunflower, tomato, pepper, chrysanthemum.

Grain plants that provide seeds of interest and to which methods and uses of the invention can be applied include oil-seed plants and leguminous plants. These include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils and chickpea.

In particular, the invention is applicable to crop plants such as those including: corn (Zea mays), canola (Brassica napus, Brassica rapa ssp.), alfalfa (Medicago sativa), rice (Oryza sativa), rye (Secale cerale), sorghum (Sorghum bicolor, Sorghum vulgare), sunflower (Helianthus annua), wheat (Triticum aestivum), soybean (Glycine max), tobacco (Nicotiana tabacum), potato (Solanum tuberosum), peanut (Arachis hypogaea), cotton (Gossypium hirsutum), sweet potato (Iopmoea batatus), cassava (Manihot esculenta), coffee (Coffea spp.), coconut (Cocos nucifera), pineapple (Ananas comosus), citrus tree (Citrus spp.) cocoa (Theobroma cacao), tea (Camellia senensis), banana (Musa spp.), avocado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beet (Beta vulgaris), oats, barley, vegetables and ornamentals.

Similarly, the invention can be applied to perennial fast growing herbaceous and woody plants, for example trees, shrubs and grasses. A non-exhaustive list of examples of tree types that can be subjected to the methods and uses of the invention includes poplar, hybrid poplar, willow, silver maple, black locust, sycamore, sweetgum and eucalyptus. Shrubs include tobacco. Perennial grasses include switchgrass, reed canary grass, prairie cordgrass, tropical grasses, Brachypodium distachyon, and Miscanthes.

### EXAMPLES

### Example 1: Preparation of Arabidopsis Plants Transformed to Express Transcription Factor RKD4 on Dexamethasone Induction

Arabidopsis thaliana (Col-0 genetic background) plants were transformed to have a two-component dexamethasone inducible expression vector to provide for transient expression of RKD4 transcription factor upon induction with dexamethasone.

Molecular Cloning

An RKD4 inducible expression construct was made by chemically synthesizing a DNA fragment encoding for the RKD4 protein (At5g53040). Restriction enzyme sequences were included at both ends of the synthetic fragment to facilitate cloning into a pOp6-OCS cassette (see Craft et al (2005) Plant J. 41(6): 899-918). This fragment was later subcloned into a binary vector containing a CaMV 35s-LhGR-N construct (see Samalova et al., (2005) Plant J. 41(6): 919-935). The final vector, pBIN-LhGR>>RKD4 was transformed into Agrobacterium tumefaciens (strain GV3101) and this new strain was used for plant transformation by floral dipping (see Clough and Bent, (1998) Plant J. 16(6): 735-743.

Seeds from flowers exposed to Agrobacterium were sown on MS media containing 50 ug/ml Kanamycin to identify transformed plants. To identify lines that were suitable for our experiments, we sowed progenies of transformed plants in MS media. Five days after germination plantlets were transferred to media containing 20 μM dexamethasone, which acts a transcriptional chemical inducer in this system. Lines suitable for controlled RKD4 expression were selected based on the formation of embryogenic structures in the root tips.

Selected lines were allowed to self-pollinate over two generations and homozygous lines were identified by selection on Kanamycin MS media.

### Example 2: Induction of Transformed Arabidopsis Plant Material and Regeneration of Epigenetic Variant Progeny Plants

Plant Materials and Growth Condition

A. thaliana seeds were sterilized with 5% bleach for 5 minutes, then washed five times with sterile double distilled water. A few drops of 0.1% agarose was added after the final washing. The seeds were then sowed into MS media containing 2% of sucrose and 8 g/L of plant agar. The seeds were stratified for four days at 4° C. to break the dormancy. After four days, the plates were moved into control environment with following conditions; 10 kLux light for 16 h, dark for 8 h; 22° C./18° C. day/night temperature; 50%/60% relative humidity at day/night. The plants were allowed to grow for six days and then transferred to soil until they reached maturity.

Somatic Embryo Regeneration

Arabidopsis plants were grown for six days in MS media, and plantlets were moved into MS media containing 20 μM dexamethasone. After six days, plants were moved to MS media (without dexamethasone) and developing embryos were dissected from somatic embryos forming in roots (named RO) or leaves (named LO) by micromanipulation and transferred to MS media until roots and leaves developed fully. Finally, plantlets were moved to soil and grown until maturity to collect dry seeds.

### Example 3: Comparison of Genetic Methylation Features Between Parent and Regenerant Plants

Leaves and root samples were collected from regenerated plants and their progeny in order to assess changes in DNA methylation and gene expression profiles. The molecular analysis was carried out using Next Generation sequencing and data were analysed using computational methods. This strategy allowed precise identification of stable changes in DNA methylation and gene expression. In addition, plants were grown in order to conduct phenotypic analyses. Changes in flowering time were assessed by growing plants in short and long day conditions. Further, enhanced resistance/susceptibility to three different pathogens was assessed: Botrytis cinerea, Hyaloperonospora parasitica and Pseudomonas syringae pv. tomato DC30000 (Pst DC3000).

Nucleic Acid Extraction

Leaf samples were collected from five individual five-weeks-old plants. The leaf samples were collected in 1.5 mL Eppendorf tubes and flash-frozen in liquid nitrogen and stored under −80° C. until further use. The samples were grounded in a mortar with the addition of liquid nitrogen to prevent sample from thawing. After the samples were completely pulverized, the genomic DNA was extracted using Qiagen Plant DNeasy kit (Qiagen). The quality and quantity of genomic DNA was checked using agarose gel electrophoresis and NanoDrop (Thermo Scientific).

The total RNA sample is extracted from leaf samples using Qiagen Plant RNeasy kit (Qiagen) following the manufacturing manuals. The quality and quantity of total RNA will be analyzed using agarose gel electrophoresis and NanoDrop (Thermo Scientific).

Methylation Analysis—Library Preparation:

Preparation of DNA libraries for bisulphite sequencing was adapted from Lister et al. (2008) Cell 133: 1-14. Libraries were constructed starting from 100 ng of purified genomic DNA using the Illumina TruSeq DNA Sample Prep kit (San Diego, Calif., USA) according to the manufacturer's instructions with the following modifications. After adapter ligation, non-methylated cytosine residues were converted to uracil using the EpiTect Plus DNA Bisulfite kit (Qiagen) according to the manufacturer's guidelines. For higher conversion efficiency the bisulphite incubation was doubled. Library enrichment was performed with the Kapa Hifi+ Uracil Hotstart Polymerase (PeqLab) and 14 PCR cycles.

Methylation Analysis—Bisulphite Sequencing:

Bisulphite sequencing was performed on an Illumina HiSeq2000 instrument. Bisulphite-converted libraries were sequenced with 2×101-bp paired-end reads and a 7-bp index read. For bisulphite sequencing, conventional A. thaliana DNA genomic libraries were analysed in control lanes. For image analysis and base calling, we used the Illumina OLB software version 1.8.

Methylation Analysis—Processing and Alignment of Bisulphite-Treated Reads:

The method adapted from Becker et al., (2011) was used. The SHORE pipeline was used to trim and filter the reads. Reads with more than 2 bases in the first 12 positions with quality score less than 3 were deleted. The reads with quality values equal to or greater than 5 were trimmed to the right-most occurrence of two adjacent bases. All trimmed reads shorter than bp were deleted. All the high quality reads were aligned to TAIR9 (http://www.arabidopsis.org) by using a modified version of the mapping tool GenomeMapper.

All alignments with the least amount of mismatches for each read were reported by GenomeMapper. However, only reads mapping uniquely to a single position were used for this study. Furthermore, all but one read were removed from further analysis if their 5′ ends aligned to the same genomic position, to account for amplification biases. A paired-end correction method was used to discard repetitive reads by comparing the distance between reads and their partner to the average distance between all read pairs. Reads with abnormal distances were removed if there was at least one other alignment of this read in a concordant distance to its partner. Finally, read counts on all cytosine sites were obtained with SHORE. The ‘scoring matrix approach’ of SHORE assigns a score to each site by testing against different sequence and alignment related features. For comparisons across lines, cytosines were accepted if at most one intermediate penalty on its score was applicable to at least one strain (score ≧32). In this case, the threshold for the other strains was lowered, accepting at most one high penalty (score ≧15). In this way, information from other strains is used to assess sites from the focal strain under the assumption of mostly conserved methylation patterns, allowing the analysis of additional sites. The methylation statistics on each single strain assumed a quality score of 25 or higher, which means no more than two intermediate penalties.

Methylation Analysis—Determination of Methylated Sites:

To minimize false-positive methylation detection, an independent binomial model to the relative proportions of converted and unconverted reads that cover cytosines in the chloroplasts were fitted. The binomial rate of false-positive methylation from the maximum likelihood was estimated separately for each library and for different bins of total read coverage:

To account for the variability in error rates in the downstream analyses, specific error models for each strain and for read-coverage bins of multiples of fivefold, yielding error rates between 0.2% and 5.0% were used. For coverage bins with too few sites for robust statistical estimation (<50), the false methylation rate from the closest sufficiently populated coverage bin was imputed. Given the estimated rates for false methylation, a genome-wide test for significant methylation of cytosines was carried out. For each site, the P value under the background model was calculated. Storey's method, an extension of the Benjamini-Hochberg stepdown procedure, to assess genome-wide significance using q values was used, and a joint false discovery rate (FDR) of 5% was used.

Methylation Analysis—Identification of Differentially Methylated Positions:

From total cytosines obtained which fulfill high-quality reads criteria in each strain, one that has significant methylation in at least one strain was chosen. Sites with statistically significant methylation differences were identified with Fisher's exact test. P values from individual tests per site were combined into single P values via conservative Bonferroni correction. Genome-wide FDRs were then estimated using Storey's method. To limit false-positive DMPs, FDR of 10% was used. The pairwise tests were performed for each RO generated plants against LO generated plants.

The same strategy was applied to identify DMPs that differed either between the RO-plants and LO-plants, or between RO/LO-plants. Count data from replicates were combined for each site, followed by pairwise Fisher's exact tests between all combinations of strains. P values for at least one differential pair were estimated using a Bonferroni correction, followed by Storey's method to assess genome-wide significance.

Methylation Analysis—Identification of Differentially Methylated Regions:

Differentially methylated regions (DMRs) were identified as described in Hagmann et al., (2015) PLOS Genetics, Volume 11, Issue 1, e1004920. First, contiguous stretches of methylation, herein referred to as methylated regions (MRs) were detected using a Hidden-Markov-Model based approach in every individual sample (Hagmann et al., (2015)). For differential analysis of MRs, all LO samples were grouped as replicates. MR segments were then tested for differential methylation between these groups.

Expression Analysis:

All RNA samples were prepared with the TruSeq RNA sample prep kit (Illumina, SanDiego, Calif.), according to the manufacturer's instructions. Samples were sequenced on a HiSeq2000 at a depth of 20-30 million reads per sample. Transcript abundance was calculated by mapping reads to the combined transcript models of the Arabidopsis reference genome using bwa. Reads were filtered to allow for only uniquely mapped reads. Differential expression was calculated using the DESeq package in R (v3.0.1).

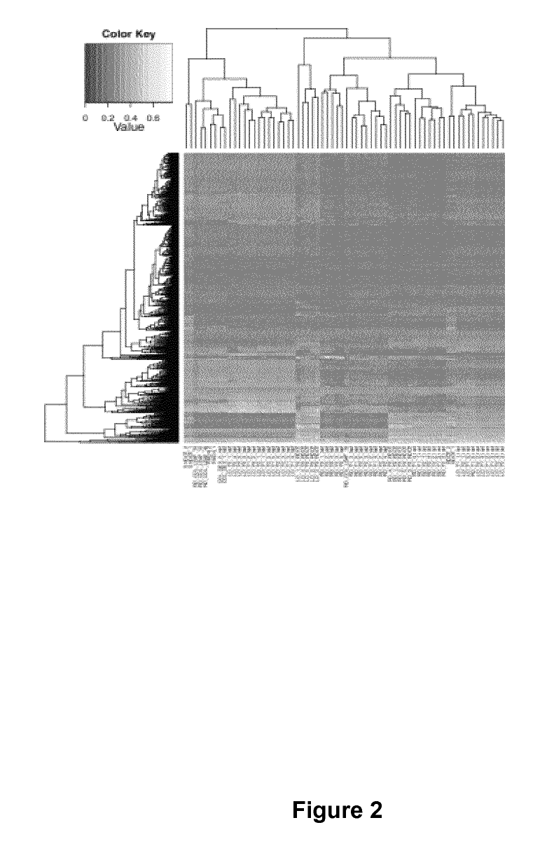

Short read sequencing of bisulphite treated genomic DNA and computational analysis identified regions of the genome that differed between parents and regenerants of Example 2. FIG. 2 is an unsupervised cluster analysis of differentially methylated regions (DMRs). The analysis shows that for the two organs tested, leaves of root-cell-regenerants (RO) contained DNA methylation features typically found in roots, whereas leaf-cell-regenerants (LO) displayed methylation features typically only found in leaves. The progenies of RO and LO plants were analysed and it was found that these DNA methylation features where stably inherited over two generations. These results show that the method of reprogramming somatic cells with zygotic or early stage transcription factors can be used to engineer a range of plants, each with specific epigenetic character, which is stably inherited by the offspring over subsequent generations.

### Example 4: Determination of the Extent of Epigenetic Variation Between Regenerant Plants

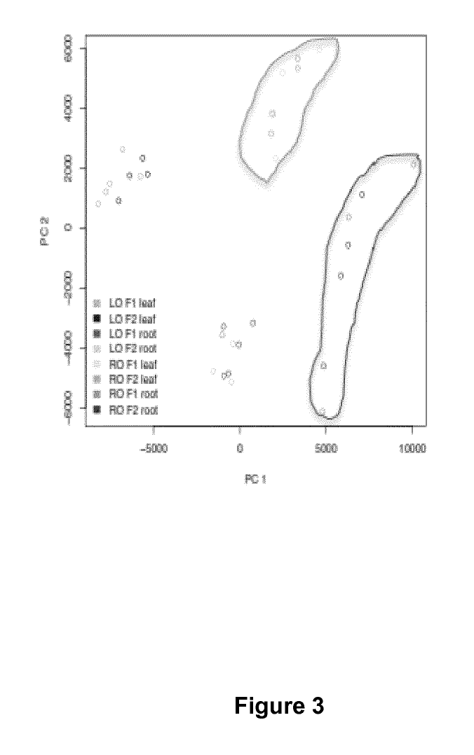

The level of novel epigenetic variation generated was assessed in the regenerant plants of Example 2. The DNA methylation profiles of each individual regenerant were analysed by a principal components analysis (FIG. 3), which revealed that whilst RO regenerants displayed highly diverse DNA methylation patterns, LO regenerants where largely uniform. These results indicate that roots contain a highly complex source of epigenetic cellular variation and that our method is able to fix and stabilize such epigenetic variation in whole plants.

### Example 5: Correlating Epigenetic Variation to Changes in Gene Expression

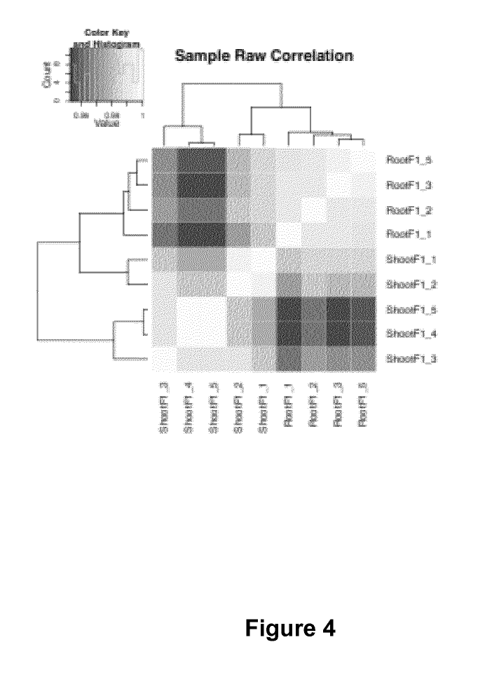

Given that individual regenerant plants have unique epigenetic character, it was verified whether these plants would have patterns of gene expression not found in the parents. RNA was extracted from leaves of parents and regenerants, and a genome-wide gene expression analysis was carried out using Next Generation Sequencing. FIG. 4 shows the unsupervised cluster analysis of whole-genome expression of plants regenerated from root (RO) and leaf (LO) tissues. Leaves of RO regenerants displayed a novel pattern of gene expression not present in the parents. A computational analysis of the gene expression data found that >400 genes were differentially expressed in leaves of RO regenerants. Further analysis revealed that 73% of these genes are known to be activated in response to pathogens and also regulate plant growth.

### Example 6: Analysis of Difference in Gene Expression Between Parents and Regenerants in Response to Stress

Changes in gene expression between parents and regenerants are associated with growth phenotypes in response to biotic stress. Parents and regenerants were grown under controlled growth conditions and both groups were subjected to infection with three different pathogens.

Plant Pathogen Infection Assays:

Arabidopsis plants were grown from 2 weeks under short day conditions (8 h light/16 h dark). Rosette leaves were infected with an inoculum of three different pathogens: Botrytis cinerea, Hyaloperonospora parasitica and Pseudomonas syringae pv. tomato DC30000 (Pst DC3000). Resistance to infection was determined after 5 days by microscopic investigation. We determined in infected leaves the size of necrosis formed after Botrytis infection, bacterial growth on leaf surfaces by Pseudomonas growth and number of spores on leaf surfaces after Hyaloperonospora infection.

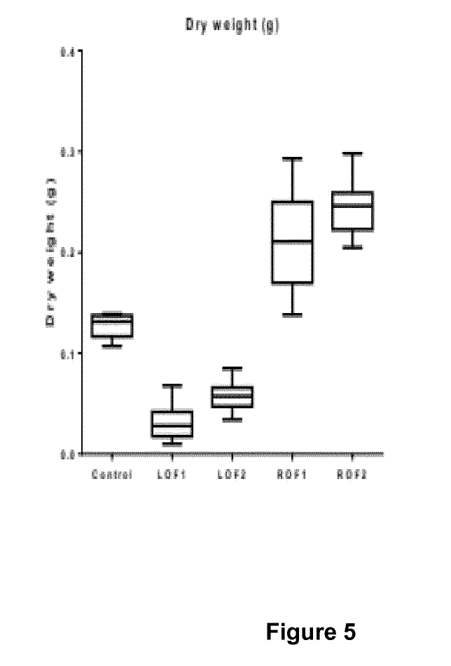

Plants were collected two weeks after infection and dry weights measured. FIG. 5 shows the results in which regenerants displayed phenotypes not previously observed in the parents. In particular, RO regenerants grew more vigorously that LO regenerants and parental lines. These new phenotypes were stable in subsequent generations. These results show how the methods of the invention generate novel beneficial phenotypic traits that can be further selected for in plant improvement programs.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method of making a plant having stable, heritable epigenetic character different from the parental plant, comprising:
a. introducing in a parental plant or plant material with an expression construct that comprises a nucleic acid sequence encoding a zygotic and/or an embryonic transcription factor;
b. regulating the expression of the transcription factor in the plant or plant material for a period of time; then
c. growing transformed plant material in culture without expressing the transcription factor;
d. identifying a regenerant plant initial, plant embryo or plantlet; and
e. growing the plant initial, plant embryo or plantlet into a mature progeny plant.

2. A method as claimed in claim 1, wherein the expression construct is introduced into the parental plant or plant material by a process of transformation; preferably using Agrobacterium.

3. A method as claimed in claim 1, wherein the plant material is an organ, a tissue, a seed or cells; optionally wherein the material is isolated from the parent plant prior to transformation of that material with the expression construct.

4. A method as claimed in claim 1, wherein prior to step (b) a whole parent plant from step (a) is grown to reproductive maturity, selfed and then grown on to set seed, wherein the plant material of step (b) is the or a seed.

5. A method as claimed in claim 1, wherein the expression construct is an inducible expression construct; optionally one part of a two-part inducible expression system.

6. A method as claimed in claim 1, wherein expression of the transcription factor is inducible for the period of time.

7. A method as claimed in claim 5, wherein the expression is inducible with a chemical; optionally wherein the chemical inducer is selected from alcohol, tetracycline, steroid, metal or pathogenesis related protein; preferably wherein the chemical is dexamethasone.

8. A method as claimed in claim 7, wherein following contacting transformed material with the chemical inducer for a period of time, the tissue or cells are transferred to a regeneration medium lacking the chemical inducer.

9. A method as claimed in claim 1, wherein the transcription factor is selected from: RKD4, BBM, LEC2 and FUS3.

10. A method as claimed in claim 1, wherein before and/or after transformation isolated tissue or cells are sorted; optionally according to one or more of tissue or cell type, protein marker, methylation profile or gene expression profile.

11. A method as claimed in claim 1, wherein the tissue or cells are from the root of the parent plant.

12. A method as claimed in claim 1, wherein the parent plant is subjected to a stress condition; optionally wherein the stress condition is abiotic.

13. A method as claimed in claim 1, wherein the epigenetic character is manifest as at least one quantitative trait.

14. A method as claimed in claim 1, wherein the epigenetic character is identifiable by DNA methylation profile.

15. A method as claimed in claim 1, wherein an ovule of the progeny plant is fertilized with pollen of the same plant and the plant then grown on to set seed.

16. A method as claimed in claim 15, wherein a seed of the plant is collected, germinated and then the resultant seedling is grown into a mature plant.

17. A method as claimed in claim 16; preferably repeated a number of times; optionally 2, 3, 4, 5 or more times.

18.-19. (canceled)

20. Plants, plant cells, tissues, organs or parts, obtainable by a method claim 1.

21. A plant-derived product obtained from a plant of claim 20.

22. A method as claimed in claim 12, wherein the stress condition is selected from one or more of photoperiod, temperature or salinity.
